(19) United States
(12) Patent Application Publication
(10) Pub. No.: US 2006/0146282 A1
(43) Pub. Date: Jul. 6, 2006

(54) Visual recognition improvement system

(75) Inventors: Seiji Kawai, Aichi-gun (JP); Hiroto Nakatani, Nagoya-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 11/300,266

(22) Filed: Dec. 15, 2005

(30) Foreign Application Priority Data
Dec. 17, 2004 (JP) 2004-366689

(51) Int. Cl. A61B 3/00; G06K 9/40

(52) U.S. Cl. 351/203; 382/254

(57) ABSTRACT

A visual recognition improvement system includes a visual noise generation device for generating and emitting noise ridden light at a predetermined intensity for an improvement of visual recognition; and a reflective device for reflectively redirecting the noise ridden light emitted from the visual noise generation device. The noise ridden light is projected toward an eye of an operator of a moving mechanism at work by using the reflective device from a plurality of directions in an eyesight of the operator.

### CROSS REFERENCE TO RELATED APPLICATION

This application is based on and claims the benefit of priority of Japanese Patent Application No. 2004-366689 filed on Dec. 17, 2004, the disclosure of which is incorporated herein by reference.

### FIELD OF THE INVENTION

The present invention generally relates to a visual recognition improvement system.

### BACKGROUND OF THE INVENTION

In recent years, a headlight control system for assisting a driver of a vehicle to recognize an object such as a pedestrian or the like without alerting the pedestrian has been proposed (refer to Japanese Patent Document JP-A-2000-203335, for example). In the disclosure of this Japanese Patent Document, the headlight control system includes a first headlight for projecting a visible light towards a front space of the vehicle and a second headlight for projecting an ultraviolet light toward a front space of the vehicle. The ultraviolet light projected from the second headlight shines on clothes worn by the pedestrian, and reflects back toward the driver of the vehicle. That is, the pedestrian is lit up by the ultraviolet light. Therefore, the driver of the vehicle can visually recognize the pedestrian ahead of the vehicle, and the pedestrian is not alerted by the ultraviolet light even when he/she sees the ultraviolet light from the second headlight.

However, reflection intensity of the ultraviolet light used in the conventional headlight control system is affected by the color and material of the clothes of the pedestrian. That is, the intensity of the reflection of the ultraviolet light varies largely depending on the color of the clothes or the like. In addition, the driver can hardly recognize a small animal such as a dog, a cat or the like that may be lit up by the ultraviolet light because their coats are not very reflective. As a result, the headlight control system conventionally used for improvement of visual recognition of the object such as the pedestrian may not provide an intended improvement of the visual recognition when the ultraviolet light is not sufficiently reflected on the object.

### SUMMARY OF THE INVENTION

In view of the above-described and other problems, the present invention provides a system that is intended for an improved visual recognition to a viewer (i.e., a driver of a vehicle or the like).

A visual recognition improvement system of the present invention uses a visual noise that has a suitable intensity for visual recognition of an object based on a threshold of visibility, and provides the visual noise for the driver while he/she is driving a vehicle from a plurality of directions towards the eyes of the driver.

The present invention is based on a research result regarding “Stochastic Resonance (SR),” disclosed in a thesis entitled “Functional stochastic resonance in the human brain: Noise induced sensitization of baroreflex system” by Hidaka et al., and published in Transactions on Bionics and Physiology Symposium Vol. 15, pp 261-264.

Stochastic resonance (SR) is an experimentally proven phenomenon that improves sensitivity of perception by statistically manipulating a small noise having an intensity below a threshold of perception for an organ of interest such as an eye, an ear or the like. Thus, SR is studied for improving human perception or a similar macro function. The area of improvement may include any macro facility such as perception, nerve control, behavioral operation or the like.

SR is more practically explained with reference to the drawings. FIG. 3A shows an illustration of a human sensory system (e.g., a nerve cell) having a non-linear function. The human sensory system generally responds to an input having an intensity greater than a threshold by yielding an output, as shown in FIG. 3B. That is, an input having an intensity below the threshold cannot be detected. Therefore, one aspect of the present invention implements SR to generate random noise having a broad frequency band for increasing the magnitude of the input and effectively improving the sensitivity of the human sensory system to detect an otherwise un-perceivable input. However, the random noise for improving the sensitivity should be carefully chosen in terms of its intensity. FIG. 3C shows a relationship between a signal noise ratio (SNR) and the random noise intensity. The relationship shown in the figure indicates that the random noise should neither be too strong nor too weak for the signal of interest to be perceived. That is, a certain intensity of the random noise, i.e., an optimum intensity, will maximize the SNR.

The SR described above has an effect for improving perception such as a visual recognition of an object or the like. However, SR does not have a subliminal effect such as an enforcement of subconsciousness which is invoked by using repetitive provision of images, sounds or the like at an un-perceivable intensity.

The visual noise generated in the above-described manner by using a light is projected toward the driver effectively improves the visual recognition of the driver of the vehicle. Further, movement of the eyes of the driver towards the upper/lower and/or right/left areas of his/her sight is covered by providing the visual noise from those directions. In this manner, the visual noise is effectively provided for the driver even when the driver's eyes are directed towards multiple directions.

According to one aspect of the present invention, the visual recognition improvement system includes reflective devices as well as a noise generation device. That is, the visual noise generated by the noise generation device is projected towards the eyes of the driver and the reflective devices or other structures in the vehicle for being directly or reflectively injected into the eyes of the driver. In this manner, the movement of the driver's eyes to various directions such as, for example, a left direction for viewing a rear-view mirror, is always covered by the visual noise.

Further, the visual noise reflectively redirected by a windshield of the vehicle is effectively injected into the driver's eyes, because the visual noise being reflectively redirected to the windshield by the reflective device does not include an unnecessary ghost image of a visual noise projecting portion of the visual recognition improvement system. In this manner, a single projection device can provide the visual noise from a plurality of directions by using a simple structure.

According to yet another aspect of the present invention, the reflective devices may include mirrors disposed on an instrument panel of the vehicle. In this manner, the reflective devices effectively reflects the visual noise emitted from the noise projecting portion towards the driver without obstructing the sight of the driver.

According to still yet another aspect of the present invention, the reflective devices may preferably be adjustable in terms of the direction of the reflection. In this manner, the drivers in, for example, various heights and positions can be covered.

According to still yet another aspect of the present invention, the visual noise may be provided from a device disposed on an inner surface of the ceiling of the vehicle. In this manner, the visual noise is effectively provided for the driver of the vehicle.

### BRIEF DESCRIPTION OF THE DRAWINGS

Other objects, features and advantages of the present invention will become more apparent from the following detailed description made with reference to the accompanying drawings, in which:

FIG. 1A shows a block diagram of a visual recognition improvement system with a representation of visual noise provided for a driver in an embodiment of the present invention;

FIG. 1B shows a block diagram of a noise generation unit described with a user interface and a system structure;

FIGS. 2A and 2B show an illustration of devices of the visual recognition improvement system disposed in a vehicle with the visual noise emitted therefrom in the embodiment;

FIG. 3A is a block diagram of a human sensory system;

FIG. 3B a diagram showing a relationship of an input-output characteristic having a threshold; and

FIG. 3C is a diagram of the visual noise showing a relationship between a signal noise ratio and a noise intensity.

### DETAILED DESCRIPTION OF THE PREFERRED EMBODIMENTS

An embodiment of the present invention is described with reference to the drawings.

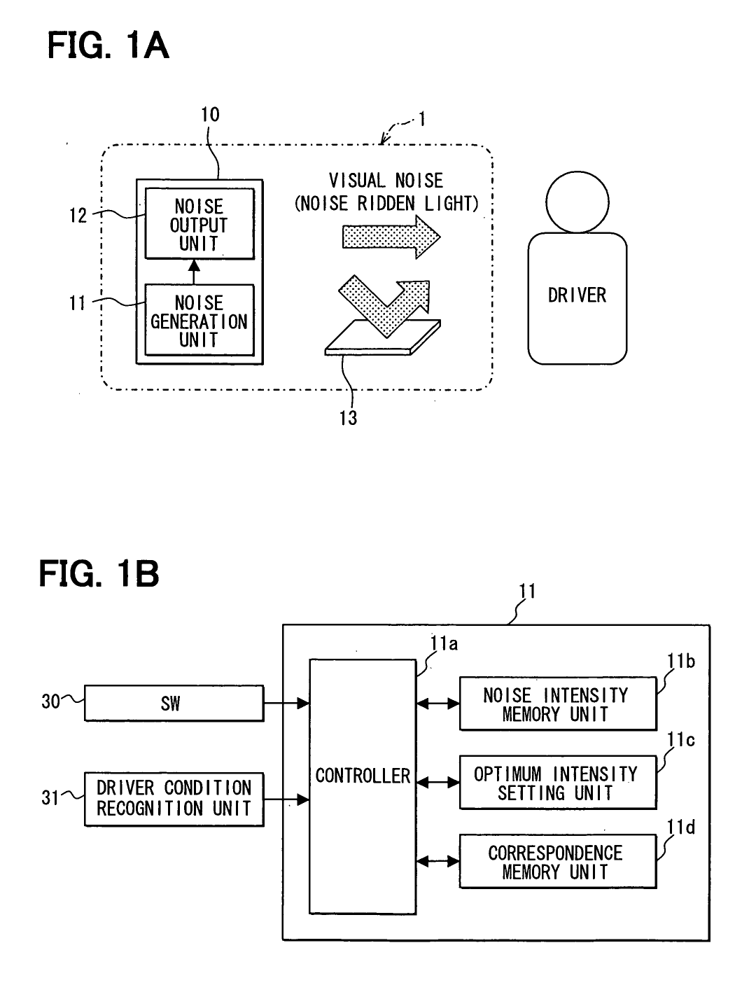

FIG. 1A shows a block diagram of a visual recognition improvement system 1 and an illustration of provision of visual noise (noise ridden light) in an embodiment of the present invention, and FIG. 1B shows a block diagram of a visual noise generation device described with a user interface and an internal system structure.

As shown in FIG. 1A, the visual recognition improvement system 1 includes a visual noise generation device 10 and a reflective device 13. The visual noise generation device 10 has a noise generation unit 11 for generating the visual noise and a noise output unit 12 for emitting the visual noise based on a signal from the noise generation unit 11.

The noise generation unit 11 includes a controller 11a, a noise intensity memory unit 11b, an optimum intensity setting unit 11c, and a correspondence memory unit 11d. That is, the noise intensity memory unit 1b memorizes a perceivable intensity of the noise as a threshold, the optimum intensity setting unit 11c sets an optimum intensity of the output noise based on the threshold memorized in the noise intensity memory unit 1b, the correspondence memory unit 11d memorizes a combination of the optimum intensity of the noise with a driver's condition derived from a drivers condition recognition unit 31, and a controller 11a for controlling the noise generation unit 11 by using the above described units 11b, 11c, 11d.

The controller 11a is coupled with operation switches 30 and the driver condition recognition unit 31. The controller 11a defines the optimum intensity of the noise for a current condition of the driver based on the combination of the optimum intensity and the driver's condition memorized in the correspondence memory unit 11d. The controller 11a also controls generation of the visual noise based on the optimum intensity determined in the above-described manner. The controller 11a sends a control signal to the noise output unit 12 for an output of the visual noise having the optimum intensity. The noise output unit 12 emits the visual noise from the noise generation unit 11 by controlling a light source based on the control signal. The light source used to emit the visual noise may be, for example, a light emitting diode (LED), a room lamp in a vehicle 2 or the like.

The visual noise used for the improvement of visual recognition by inducing the stochastic resonance (SR) is preferably wideband noise having no intensity peak at any range of frequency. That is, the stochastic resonance in visual recognition is induced by using noises such as, for example, a random noise having constant intensity at the visible band and/or a 1/f noise having intensity inversely proportional to frequency.

However, the visual noise having a wide range of frequency band may not be having a sufficient intensity nor an appropriate intensity relative for suitably enhancing the visual recognition by inducing the SR. In this case, the signal noise ratio (S/N ratio) in the output signal is decreased.

Therefore, the intensity of the visual noise used for visual recognition improvement should carefully be chosen. That is, for example, the optimum intensity of the visual noise is determined by an experiment to be stored in the visual recognition improvement system 1 for use in the visual noise generation in the noise generation device 10. The control signal for generating the visual noise is outputted based on the stored intensity.

Difference of the optimum intensities among individual drivers are accommodated by receiving feedback from each driver through the operation switch 30 while the controller 11a gradually increases the intensity of the visual noise outputted from the noise output unit 12. The optimum intensity of the visual noise suitable for each driver can be captured and stored in the noise intensity memory unit 11b by having a feedback as a threshold of the noise intensity from the driver at the moment of recognition of the visual noise through the operation switch 30. The optimum intensity setting unit 11c sets the optimum intensity of the visual noise for outputting the control signal of visual noise generation at the intensity of, for example, approximately 100% of the threshold intensity for the random noise, and approximately 69% of the threshold intensity for the 1/f noise. The proportion of the noise intensity for a sufficient signal noise ratio may be, for example, derived from an experiment. The percentage of the intensity against the threshold intensity may be variably controlled according to the situation of use of the visual recognition improvement system 1.

The driver condition recognition unit 31 may be, for example, a heart rate sensor for detecting a heart beat rate of the driver at the wheel. The detected heart beat rate may be used as an index of arousal. The heart beat rate may be detected by using devices such as a detector in a shape of a wrist watch, a device in a steering wheel for detecting the heart beat rate through blood pressure, a detection device in a seat or the like.

The controller 11a may preferably increase the intensity of the visual noise by controlling the noise output unit 11 when the arousal of the driver detected by the driver condition recognition unit 31 is decreasing.

The reflective device 13 includes a mirror for reflecting the visual noise from the visual noise generation device 10. The mirrors in the reflective device 13 may be manually adjusted to have a suitable angle for reflection.

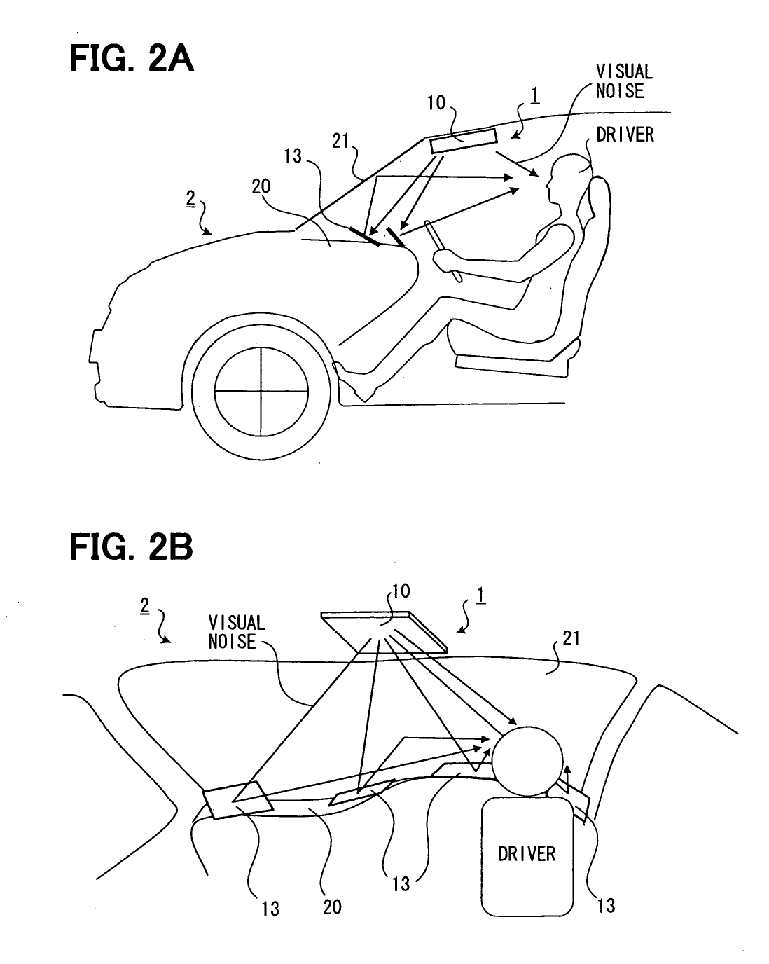

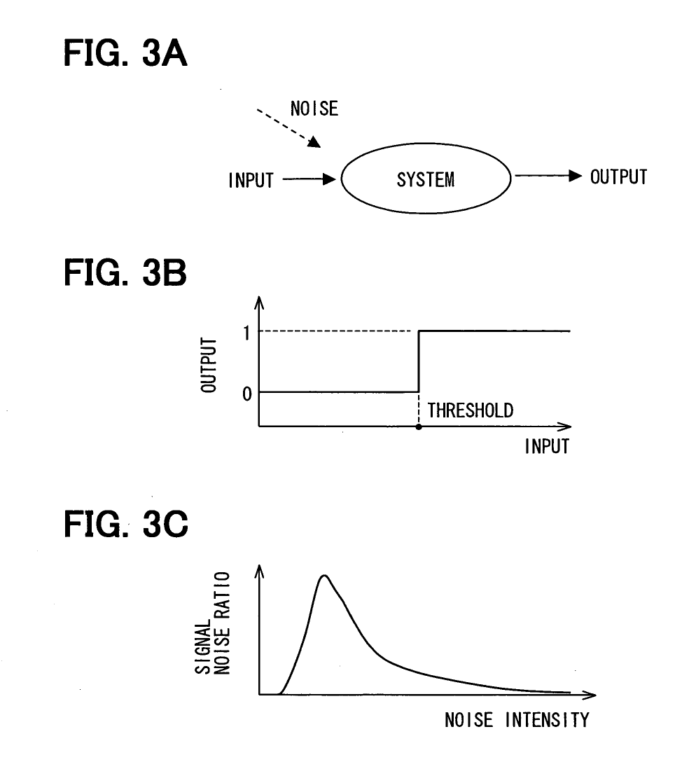

FIGS. 2A and 2B show an illustration of the visual recognition improvement system 1 disposed in the vehicle 2 with the visual noise emitted from the visual noise generation device 10. FIG. 2A shows visual noise emission in the vehicle 2 shown in a side view of the vehicle 2, and FIG. 2B shows visual noise emission in a room of the vehicle 2 illustrated as a view from a rear to a front.

The one visual noise generation device 10 is disposed on the ceiling of the vehicle 2 in a portion in front of a head of the driver as shown in FIG. 2A. The four reflection devices 13 are disposed on an instrument panel 20 as shown in FIG. 2B. In this manner, the visual noise generation device 10 at the described position can effectively provide the visual noise to the eyes of the driver and the reflective devices 13 even when the device 10 is disposed only at one place in the vehicle 2. The visual noise directly provided to the eyes of the driver designed to enter into the eyes when the driver is looking right front of the vehicle 2.

The reflective devices 13 are disposed at various positions so that the reflection of the visual noise enters into the eyes of the driver when the driver moves his/her sight away from the right front for confirmation of the rear view mirrors or the like. That is, the reflective devices 13 at right-most and left-most ends of the instrument panel 20 may be used, for example, to reflect the visual noise when the driver is looking into the rear view mirrors by horizontally moving the sight in a relatively large amount, and the reflective devices at a center of the instrument panel 20 may be used, for example, to reflect the visual noise when the driver is looking for a traffic light, a road sign or the like by vertically moving the sight in a small amount.

The visual noise redirected by a front windshield 21 may effectively enters into the eyes of the driver without having a ghost image of the visual noise generation device 10 because of a reflection by an intermediate reflective device 13 in a path of the visual noise prior to a reflection by the front windshield 21.

The mirrors in the reflective device 13 may be held by using ball joints or the like for variable adjustment of the reflection angle. In this manner, various shapes and positions of the drivers can suitably be covered by the visual noise reflected by the mirrors in the reflective device 13.

The visual recognition improvement system 1 of the present invention may effectively provide the following effects for the user of the system. That is, (a) the visual noise directly emitted towards the driver can improve the visual recognition when the driver is looking right in a front space of the vehicle 2, and the visual noise redirected by the mirrors of the reflective device 13 can improve the visual recognition when the driver is looking sideways for checking side mirrors or the like. In addition, (b) the visual noise generation device disposed only at one position can provide the visual noise from various directions towards the driver by using a simple system structure. Further, (c) the visual noise can effectively provided for the driver when the reflective device 13 including mirrors is disposed on the instrument panel 20. Furthermore, (d) the visual noise can appropriately be provided for the driver when the mirrors in the reflective device 13 are manually controlled according to the shape and/or positions of the driver. Furthermore, (e) the visual noise generation device 10 in a front portion of the ceiling of the vehicle 2 relative to the head of the driver can effectively provide the visual noise for the driver and/or the reflective device 13.

Although the present invention has been fully described in connection with the preferred embodiment thereof with reference to the accompanying drawings, it is to be noted that various changes and modifications will become apparent to those skilled in the art.

For example, the reflective device 13 may be disposed on a steering post or on the ceiling so that the device 13 does not interfere with the sight of the driver.

Further, the visual noise generation device 10 may be disposed at a plurality of positions instead of having the reflective devices 13 in plural numbers. Furthermore, plural reflection devices 13 may be used in combination with the plural noise generation devices 10.

Such changes and modifications are to be understood as being within the scope of the present invention as defined by the appended claims.

What is claimed is:

1. A visual recognition improvement system comprising:
a visual noise generation device for generating and emitting noise ridden light having a predetermined intensity based on a threshold of perceptibility of a visual noise in the noise ridden light,
wherein the noise ridden light is projectively supplied for an eye of an operator of a moving mechanism at work in pursuit of an improvement of visual recognition.

2. A visual recognition improvement system comprising:
a visual noise generation device for generating and emitting noise ridden light; and
a reflective device for reflectively redirecting the noise ridden light emitted from the visual noise generation device,
wherein intensity of a visual noise in the noise ridden light is determined based on a threshold of perceptibility of the visual noise, and
the noise ridden light is projectively supplied for an eye of an operator of a moving mechanism by using the reflective device from a plurality of directions within an eyesight of the operator at work in pursuit of an improvement of visual recognition.

3. The visual recognition improvement system according to claim 2,
wherein the reflective device redirects the noise ridden light towards the eye of the operator in a path that reflectively passes a structure of the moving mechanism.

4. The visual recognition improvement system according to claim 3,
wherein the reflective device includes a mirror, and
the reflective device is disposed on an instrument panel of the moving mechanism.

5. The visual recognition improvement system according to claim 4,
wherein the reflective device is adjustably disposed in an arbitrary direction in terms of reflective redirection of the noise ridden light.

6. The visual recognition improvement system according to claim 5,
wherein the visual noise generation device is disposed on a ceiling of the moving mechanism relatively forwardly to a head of the operator.

7. The visual recognition improvement system according to claim 6,
wherein the moving mechanism is a vehicle and the operator is a driver of the vehicle.
